(19) United States
(12) Patent Application Publication
(10) Pub. No.: US 2026/0256902 A1
(43) Pub. Date: Sep. 3, 2026

(54) A VACCINE FOR PROTECTING A PREGNANT SWINE AGAINST AFRICAN SWINE FEVER

(71) Applicant: INTERVET INC., Rahway, NJ (US)

(72) Inventors: Erwin VAN DEN BORN, Wageningen (NL); Jui JOSHI, Beuningen (NL)

(73) Assignee: INTERVET INC., Rahway, NJ (US)

(21) Appl. No.: 19/162,985

(22) PCT Filed: Mar. 15, 2024
(86) PCT No.: PCT/EP2024/056974

§ 371 Date: Sep. 8, 2025

(30) Foreign Application Priority Data
Mar. 16, 2023 (EP) 23162452.9

(51) Int. Cl. A61K 39/12; A61K 9/00; A61K 39/00; A61P 31/20; C12N 7/00

(52) U.S. Cl. CPC A61K 39/12; A61K 9/0019; A61P 31/20; C12N 7/00; A61K 2039/5254; A61K 2039/542; A61K 2039/545; A61K 2039/552; C12N 2710/12021; C12N 2710/12034

(57) ABSTRACT

The invention pertains to a live attenuated African swine fever virus Georgia 2007 (ASFV-G) Δ9GL/ΔUK strain for use in a vaccine for protecting a pregnant swine against an infection with African swine fever virus (ASFV) by administering the vaccine comprising the live attenuated ASFV-G-Δ9GL/ΔUK strain to the pregnant swine.

### FIELD OF THE INVENTION

African swine fever virus (ASFV) is one of the most important disease-causing pathogen affecting the domestic swine population globally. The present invention pertains to a vaccine to protect swine against an infection with ASFV, in particular the vulnerable group of pregnant swine.

### BACKGROUND OF THE INVENTION

As described in the review article by A.C. Urbano et al. (Emerging Microbes & Infections, 2022, Vol. 11, pp 2021-2033), African swine fever (ASF) is a highly infectious and severe haemorrhagic viral disease of pigs, endemic to South-Saharan Africa (24 genotypes based on the sequence of the c-terminus of the p72 surface antigen) and the island of Sardinia in Italy (p72 genotype 1). The transcontinental spread of ASF occurred on at least three separate occasions, most significantly to Georgia, in 2007, where it spread from the Black Sea port of Poti across the Caucasus region into the Russian Federation (RF) and Eastern Europe. The following decade saw the disease become epizootic in the RF, and by 2018 it had spread as far West as Belgium and East to the People's Republic of China, quickly taking over most of Southeast Asia and Oceania. All isolates found in these regions are related to the Georgia 2007 isolate, commonly referred to as ASFV Georgia 2007 or ASFV-G (U.S. Pat. No. 9,808,520). Since 2018, the epidemiological situation of ASF has continued to deteriorate; in January 2022 ASF virus reappeared on the Italian mainland. Several reoccurrences have since been reported in China, the RF, Moldova, and Ukraine, and North Macedonia reported their first occurrence, as did Thailand, one of the few countries in the region that had remained unscathed. The virus also appeared in 2021 in the Dominican Republic and Haiti, constituting the first diagnosis of ASFV in more than 40 years in the Western hemisphere. These recent events highlight an extremely disconcerting pattern of continuous spread, exacerbated by the fact that in many of these regions small-scale and semi-industrial farms account for the majority of pig production. As such, outbreaks carry severe socio-economic consequences, causing devastation of rural livelihoods dependent on livestock production, and threatening overall market stability and food security, as well as severely affecting animal welfare.

The African swine fever virus (ASFV) is a large, double-stranded nucleocytoplasmic DNA arbovirus, the only member of the Asfarviridae family. Virions have a diameter of around 250 nm and consist of a central nucleoid enclosed by an icosahedral protein capsid (or core shell), an internal lipoprotein membrane (or inner envelope), an icosahedral protein outer capsid, and an external lipoprotein envelope (or outer envelope) that is obtained when the virus buds out through the plasma membrane. Both intracellular and extracellular viral forms are infectious. Its natural host range is limited to soft-bodied ticks of the genus Ornithodoros and members of the family Suidae, where it replicates mainly in cells of the mononuclear phagocytic system, resident macrophages, and specific reticular cells. The virus is endemic in African wild pigs. In domestic pigs and wild boar, however, clinical signs vary considerably, and the individual out-come can range from fatal to subclinical. Depending on the virulence of the strain involved, a graded series of forms occurs, with lethality ranging from 100% (per-acute form) to <30% (chronic form) of the infected animals. The genotype II strain currently affecting Europe and Asia is highly virulent, causing the acute form of the disease, although there is evidence that some reduced-virulence isolates may be circulating among wild boar in the Baltic States and domestic pigs in China, with reports of both naturally mutated genotype II low virulent strains and genotype I low virulent epidemic strains detected in the field.

Protective immunity against ASFV is still poorly understood. There are several vaccine approaches known in the art, but this has not yet led to a commercially available vaccine. The first approach described in the art is vaccinating with an inactivated vaccine. Virus inactivation is an established approach to vaccine production, relatively straightforward to achieve and importantly, with a higher safety profile when compared to live vaccines. The inactivation process negates reversion to a virulent phenotype and renders vaccine viruses nontransmissible, the two major drawbacks of attenuated vaccines. Also, it is believed that the safety for more vulnerable patient groups such as very young animals and pregnant animals is less of a risk. The animals in the latter group go through significant physiological changes and are thus in a stressful metabolic status. Inactivation however, does not necessarily produce a vaccine that elicits protective immunity. Attempts at immunization of pigs with a variety of inactivated ASF antigens, did not lead to sufficient protection even though in some cases it was capable of inducing a serological immune response (Cadenas-Fernández, et al. Vaccines, 2021, 9, 242; doi.org/10.3390/vaccines9030242).

Another approach is the use of subunit, DNA and virus vectored vaccines. These vaccines show promise and several candidates have been shown to induce specific humoral and/or cellular immune responses which appear to confer partial to full protection. However, the different nature of the immunization protocols used in these studies, including the type of vaccine, vaccination strategy and challenge model, makes results difficult to compare. Further work will be needed to identify which immune mechanisms need to be triggered to confer complete, lasting protection, which antigens (or combination of) should be included in a potential vaccine, and the most appropriate delivery method (Urbano, supra).

Live attenuated vaccines, although inherently less safe than the above mentioned vaccines, are the most promising ASF vaccine candidates. These vaccines circumvent a key issue presented by both inactivated, subunit, vector and DNA vaccines. Because they can successfully replicate within the host, they mimic natural infection thereby triggering both humoral and cellular pathways, and typically do not require adjuvants. Additionally, some live attenuated vaccines have been shown to elicit mucosal IgA antibodies, an important feature for vaccines administered via the oral route (oral immunisation is a practical convenience for vaccines aimed at the wild boar population). That being said, these vaccines also pose a risk, as they may regain pathogenicity (i.e. revert to virulence), causing the spread of disease, and they have the potential to cause post-vaccination reactions and side effects, in particular in vulnerable animals. Three main strategies have been employed in the generation of live attenuated ASF vaccines, attenuation by cell passage, screening for naturally attenuated strains, and deletion of virulence-associated genes. To overcome some of the safety issues presented by live attenuated vaccines, in particular residual virulence, attempts have also been aimed at further deletion of virulence-associated genes in naturally attenuated strains, or adaptation to heterologous cell lines of gene deleted viruses. Depending on the immunogenicity of the deleted genes, these candidates may also be suitable for a vaccination protocol known as ‘DIVA’: differentiating naturally infected from vaccinated animals.

As indicated in Urbano (supra), in the meantime about 10 promising live attenuated vaccines are available, developed in 2015-2022. In particular NH/P68, OURT88/3, Lv17/WB/Rie1, BA71ΔCD2v, HLJ/18-7GD, ASFV-G-ΔI177L, ASFV-G-ΔI177L/ΔLVR, SY18ΔI226R, ASFV-G-ΔA137R and ASFV-G-ΔE184L are indicated to be available as live ASF vaccines. Of these, the most promising live attenuated vaccine candidate to date is the ASFV-G-ΔI177L strain that was engineered by the U.S. Department of Agriculture's (USDA) Agricultural Research Service (ARS). It can be administered by the intramuscular and oronasal route, inducing robust sterile immunity against challenge with the virulent parental ASFV Georgia 2007 isolate, involved in recent outbreaks throughout Europe and Asia, and in particular this vaccine strain proved effective in follow up field trials against the virulent Vietnamese strain TTKN/ASFV/DN/2019. Since 2022 the ASFV-G-ΔI177L stain is on the market in Vietnam through a conditional license. The development of derivative strains of ASFV-G-ΔI177L is also in progress. For example, ASFV-G-ΔI177L/ΔLVR replicates efficiently in a stable porcine epithelial cell line and maintains the same level of attenuation, immunogenic characteristics, and protective efficacy in challenge studies. Other derivatives that are tested as experimental vaccines are ASFV-G-Δ9GL, ASFV-G-ΔMGF, ASFV-G-Δ9GL/ΔUK, ASFV-G-ΔI177L and ASFV-G-ΔI177L/ΔLVR, all engineered by the U.S. Department of Agriculture's Agricultural Research Service.

### OBJECT OF THE INVENTION

It is an object of the invention to provide for an ASFV vaccine that is safe for administration to a pregnant swine, an animal that due to its highly demanding metabolic status is particularly vulnerable for infections with a pathogenic microorganism, in particular leading to a loss of viable offspring. This can be expressed as the loss of piglets that could have been born alive and are able to reach an age of more than two weeks, in particular: able to reach an age of at least 15-21 days of age. The object is in particular to avoid any losses in the number of piglets of over 50%: i.e. more than 50% of the piglets carried are born still, or do not survive more than two weeks due to the ASFV vaccine being administered to the mother animal.

### SUMMARY OF THE INVENTION

In order to meet the object of the invention, it was found that a vaccine comprising a live attenuated African swine fever virus Georgia 2007 (ASFV-G) Δ9GL/ΔUK strain, which is known to be protective against an infection with ASFV, is safe for administration to a pregnant swine. In particular, loss of viable piglets may be very low, and can be as low as 10% which is within the typical (natural common) loss range of 5-25% for healthy swine.

To the inventors this came as a surprise, in particular since the loss of viable piglets for what is commonly regarded as the most promising ASF vaccine candidate, viz. ASFV-G-ΔI177L, can be as high as 90%. Although a loss of 50% of viable offspring might, under certain circumstances, still be acceptable for an ASF vaccine, in particular given the fact that the survival of the mother animals is crucial for a swine facility, any value above 50% is deemed unacceptable for a commercial ASF virus vaccine. Still, a lower loss such as 40%, 30%, 25%, 20%, 15%, 10%, 5% or any loss that is not higher than a loss in negative control animals (i.e. a suitable control group of healthy pregnant swine that has not been administered the ASF vaccine) is particularly preferred.

Next to a a live attenuated African swine fever virus Georgia 2007 (ASFV-G) Δ9GL/ΔUK strain for use in a vaccine for protecting a pregnant swine against an infection with African swine fever virus (ASFV) by administering the vaccine comprising the live attenuated ASFV-G-Δ9GL/ΔUK strain to the pregnant swine, the invention also pertains to the use of a live attenuated African swine fever virus Georgia 2007 (ASFV-G) Δ9GL/ΔUK strain for the manufacture of a vaccine for the protection of a pregnant swine against an infection with African swine fever virus, and to a method for the protection of a pregnant swine against an infection with African swine fever virus by administering to the pregnant swine a vaccine comprising a live attenuated African swine fever virus Georgia 2007 (ASFV-G) Δ9GL/ΔUK strain.

### Definitions

A swine is an animal that belongs to the family of Suidae, in particular a porcine animal raised by man, for example to be a feeder pig, raised for seedstock, or raised for slaughter, and wild boars.

An ASFV strain is an ASFV Georgia 2007 (ASFV-G) strain if it is the reference isolate Georgia 2007 (GenBank FR682468.2), as referred to in U.S. Pat. No. 9,808,520 and in O'Donnel et al. in the Journal of Virology, January 2017, Volume 91, Issue 1, pp 1-18; doi 10.1128/JVI.01760-16), or a natural or recombinant variant of this reference isolate. Preferably the nucleotide sequence identity of an ASFV Georgia 2007 strain for use in the invention is at least 99%, more preferably at least 99.1, 99.2, 99.3, 99.4, 99.5, 99.6, 99.7, 99.8, 99.9% or even higher, when aligned to the full length of FR682468.2. Current available molecular data derived by using standardized genotyping procedures have indicated that only this ASFV variant is present in Eastern and Central Europe after the outbreak in Georgie in 2007 (Gallardo et al. Genetic Variation among African swine fever Genotype II Viruses, Eastern and Central Europe. Emerg Infect Dis. 2014 September; 20(9): 1544-1547; doi: 10.3201/eid2009.140554).

For making the nucleotide sequence alignments, the NCBI's Blast™ computer program is used (http://blast.ncbi.nlm.nih.gov/Blast.cgi), selecting the options ‘blastn’ and “Align two or more sequences” with standard settings and default parameters, and whereby the sequence of FR682468 is selected as the subject (also known as “the target”).

ASFV-G-Δ9GL/ΔUK is a variant of ASFV-G in which the 9GL and UK genes (viral gene numbers B119L and DP96R, respectively), have been functionally disabled through deletion of at least part of these genes, such that they can no longer be expressed at a normal level (i.e. at the level of the unaltered parent strain), in particular no longer expressed at all. As is commonly known, for this typically not the complete gene is deleted, since this also brings with it the risk of interfering with the transcription of the two flanking genes in the ASFV genome, and therewith actually deleting three genes instead of one. ASFV-G-Δ9GL/ΔUK does not exclude any further mutations, either spontaneously or recombinantly, such as for example specific mutations aimed at arriving at a strain suitable for differentiating between infected and vaccinated animals.

A vaccine is a constitution suitable for application to an animal, having an acceptable safety, comprising one or more antigens in an immunologically effective amount, i.e. capable of stimulating the immune system of the target animal sufficiently to induce an immune response, such as antibodies, against the antigens and therewith against the corresponding naturally occurring antigens and therewith potentially the naturally occurring pathogen, typically combined with a pharmaceutically acceptable carrier (i.e. a biocompatible medium, viz. a medium that after administration does not induce significant adverse reactions in the subject animal, capable of presenting the antigen to the immune system of the host animal after administration of the vaccine) such as a liquid containing water and/or any other biocompatible solvent or a solid carrier such as commonly used to obtain freeze-dried vaccines (based on sugars and/or proteins), optionally comprising immunostimulating agents (adjuvants), which upon administration to the animal induces an immune response that is able to protect the animal against a (post-vaccinating) infection. For any vaccine, the requirement of an acceptable safety is as important as efficacy regarding protection.

To protect an animal against an infection with ASFV means aiding in preventing, ameliorating or curing a pathogenic infection with ASFV, or aiding in preventing, ameliorating or curing a disorder arising from that infection, for example to prevent or reduce one or more clinical signs resulting from the infection with ASFV, preferably preventing death of the animal as a result of the infection with ASFV. For a pregnant animal, the foetuses carried by the animal (i.e. any unborn animal in the stages of prenatal development) are regarded a part of the pregnant animal.

Viable piglets are piglets born alive from a sow and able to reach an age of more than two weeks, in particular reach an age of at least 15-21 days.

### FURTHER EMBODIMENTS OF THE INVENTION

In a first further embodiment of the live attenuated ASFV-G-Δ9GL/ΔUK strain for use as a vaccine in protecting a pregnant swine against an infection with African swine fever virus (ASFV) by administering the vaccine comprising the live attenuated ASFV-G-Δ9GL/ΔUK strain to the pregnant swine, the vaccine is administered to the pregnant swine in the second half of gestation. Although earlier vaccination is feasible for protection, the second half of gestation is believed to be more critical for an ASFV infection and safety of a vaccine. It was found that a live attenuated ASFV-G-Δ9GL/ΔUK strain is safe for administration at this stage of gestation and even in the last third of gestation, most notably in a period of 5-20 days before the expected date of farrowing of the pregnant swine, for example at only 15 days before the expected date of farrowing.

In another further embodiment the vaccine is administered either once during gestation to the pregnant swine, or is administered in a prime- and boost regimen during gestation. In a prime- and boost regiment, the two administrations are provided during the same gestation, but are separated in time by 1-12 weeks, typically by 2-10, 2-8, 2-6 or by 2-4 weeks.

In again another further embodiment the vaccine is administered intramuscularly, intradermally or orally.

In yet again another further embodiment the vaccine is administered with a dose of the ASFV-G-Δ9GL/ΔUK strain of at least 10² TCID₅₀ per administration, for example a dose above 103, 104 or 105 TCID₅₀ up to about 10⁶ TCID₅₀ per administration.

The invention will now be further explained using the following specific examples.

### EXAMPLES

Example 1 is an experiment for testing the safety of a protective live attenuated ASFV-G vaccine strain in pregnant sows.

Example 2 is another experiment wherein two further protective live attenuated ASFV-G vaccine strains are tested for their safety in pregnant sows.

Example 3 is an experiment to confirm the efficacy of the live attenuated ASFV-G vaccine strain.

### Example 1

Example 1 is an experiment for testing the safety of the protective live attenuated ASFV-G vaccine strain ASFV-G-ΔI177L in pregnant sows. As is known from literature, this strain is highly effective in protecting swine against an infection with ASFV (Borca et al, Journal of Virology, April 2020, Volume 94, Issue 7, pages 1-18; doi 10.1128//JVI.02017-19) and safe for administration to swine (Tran et al. in Viruses 2022, 14: Evaluation of the Safety Profile of the ASFV Vaccine Candidate ASFV-G-ΔI177L).

### Object of the Study

The goal of this experiment was to establish the safety of strain ASFV-G-ΔI177L for administration to pregnant swine.

### Study Design

Four ASFV-free and ASFV antibody-free pregnant sows were available for this study. At 100 days in gestation (i.e. approximately 2 weeks before farrowing) two pigs received 2 ml of a vaccine containing ASFV-G-ΔI177L intramuscularly (IM) in the right side of the neck at a dose of 1.5×10² TCID₅₀. Two sows served as unvaccinated controls. They received PBS similar to the vaccinated animals. Animals were monitored daily from the day before vaccination onwards for ASF-specific clinical signs. Blood samples (from tail vein) were collected at 4 days post vaccination (dpv), 11 dpv, 23 dpv, 32 dpv, while temperature was monitored daily from the day prior to inoculation onwards. Upon birth, the health status of the piglets was monitored and recorded. All the live piglets were weighed around 3-4 days after birth as well as at 16-17 days after birth. Piglets were monitored daily for ASF-specific clinical signs until the end of the study.

### Results

### Health Status of the Mother Animals

Rectal temperatures were normal (between 37.5° C. and 39° C.) and not significantly different for vaccinated versus control animals. The control animals did not exhibit any clinical signs of ASF. In the second week post vaccination one of the vaccinated animals showed slight signs of ASF, but these subsided gradually and disappeared. With PCR, no ASFV could be detected in blood samples obtained from the control animals, whereas the vaccinated animals showed virus in their blood at all timepoints.

Table 1 shows significant differences in the reproductive performance of vaccinated and control sows. In case of vaccinated sows, 43% of the piglets were born dead as compared to 17% for control sows. During the experiment, while all the live born piglets of the control sows survived into the third week of age, only 4 of the piglets born to the vaccinated sows survived this period. In addition, all surviving piglets of the vaccinated sows presented ASF-specific clinical signs and were euthanized on reaching the humane endpoint (HEP). This means that overall, there was a loss of more than 90% in viable piglets due to the vaccination with ASFV-G-ΔI177L.

TABLE 1
reproductive performance of mother animals

| | Vaccinated | control |
|---|---|---|
| Total number of piglets | 41 | 46 |
| Normal piglets at birth | 14 | 36 |
| Weak piglets at birth | 9 | 2 |
| Dead piglets at birth | 18 | 8 |
| Alive piglets at end of trial | 4 | 38 |
| Loss of viable piglets | 90% | 17% |

### Health Status of the Piglets

The rectal temperatures of the piglets born from control animals were normal throughout the experiment, ranging from 38.7 to 40° C., whereas this was up to 41° C. in the piglets born from vaccinated animals. With regard to weight gain, piglets of the control sows showed on average a 2.9-fold increase in weight when weighed at days 3-4 and day 17-18 post farrowing, while in case of the vaccinated sows surviving animals showed on average a 2.2-fold increase in weight.

The control piglets were all healthy throughout the experiment. Of the vaccinated animals, all piglets born alive showed ASF-related clinical signs, and ASFV infection was confirmed by PCR. A A1177L-specific PCR performed on DNA extracted from blood of pregnant sows and their representative piglets indicated that the vaccine strain was transmitted vertically from the pregnant sows to their piglets.

### Conclusion

By comparison of the health status of the sows (and piglets born alive from these sows), in particular since the loss of viable offspring is (significantly) over 50%, it can be concluded that the vaccine strain ASFV-G ΔI177L is not safe for administration to pregnant swine.

### Example 2

Example 2 is another experiment wherein two further protective live attenuated ASFV-G vaccine strains were tested for their safety in pregnant sows. The first vaccine candidate was derived from the Lv17/WB/Rie1 strain (WO 2020/049194). In particular, this first vaccine comprises a mutant indicated as Lv17/WB/Rie1-ΔCD, which mutant comprises the additional gene deletions ΔEP153R and ΔEP402R (see Petrovan et al, Journal of Virology, January 2022, Volume 96, Issue 1, pp 1-19). The parent strain Lv17/WB/Rie1 is safe and efficacious for use in swine (Urbano, supra) and the double knock out mutant strain is also known to be (inherently safe and) efficacious for the use in swine (European Patent Application No. EP22462011.1 for “Attenuated African swine fever virus and use thereof in vaccine compositions”, filed in the name of Intervet International BV, Consejo Superior de Investigaciones Cientificas (CSIC), Állatorvostudományi Kutatóintézet, Universidad Complutense de Madrid, and Eurofins Ingenasa SA, on 22 Nov. 2022 at the Hungarian Intellectual Property Office). The second strain is known from literature (i.a. Urbano, supra) to be protective and safe for vaccinating swine.

The second vaccine candidate tested was ASFV-G-Δ9GL/ΔUK (U.S. Pat. No. 9,808,520). This strain is also known from literature (i.a. Urbano, supra) to be protective and safe for vaccinating swine.

### Object of the Study

The goal of this experiment was to establish the safety of these two further ASFV-G strains for administration to pregnant swine.

### Study Design

Six ASFV-free and ASFV antibody-free pregnant sows were available for this study. At around 100 days in gestation (i.e. approximately 2 weeks before farrowing) two sows received 2 ml of vaccine comprising strain Lv17/WB/Rie1-ΔCD intramuscularly (IM) in the right side of the neck at a dose of 10¹ TCID₅₀. Two sows received a corresponding vaccine containing the candidate vaccine strain ASFV-G-Δ9GL/ΔUK IM in the right side of the neck at a dose of 104 TCID₅₀. The remaining two sows served as non-inoculated controls. Animals were monitored from vaccination onwards for ASF specific clinical signs. Blood samples were collected at 3 days post vaccination (dpv), 10 dpv, 21 dpv and 31 dpv, while temperatures were monitored daily from day of inoculation onwards. Upon birth, the health status of the piglets was monitored and recorded. In case of any still born or dead piglets, a blood sample was taken, whenever possible before the animal was disposed. All the live piglets were weighed 5 days after birth as well as 19 days after birth. Piglets were monitored daily for ASF-specific clinical signs till the end of the study at 22 days after birth.

### Results

### Health Status of the Mother Animals

Rectal temperatures were normal (between 37.5° C. and 39.5° C.) for the control animals and the animals that received ASFV-G-Δ9GL/ΔUK, whereas the other animals vaccinated with Lv17/WB/Rie1-ΔCD had a continuous increase in temperature and showed signs of ASF from day 4 post vaccination and onwards. Both the sows in this group were euthanized on the 10^{th} day post vaccination. The piglets from these sows were either born prematurely as they were in the process of abortion or were taken from the womb at euthanasia. The control animals and one of the animals vaccinated with ASFV-G-Δ9GL/ΔUK did not exhibit any clinical signs of ASF. The other ASFV-G-Δ9GL/ΔUK-vaccinated animal showed mild signs of ASF. With PCR, no ASFV could be detected in the control animals, whereas the vaccinated animals showed virus in their blood at all timepoints.

Regarding reproductive performance, the sows used in the study had a healthy breeding history with overall more than 80% of viable piglets born to them. In this study, however, significant differences were observed in the reproductive performance of sows vaccinated with the two different candidate vaccine strains. Piglets were born prematurely 8 to 10 days before the expected due date in case of sows vaccinated with the Lv17/WB/Rie1-ΔCD strain. Both these sows began to abort piglets, which were weak, and underdeveloped with no chance of survival. These piglets were euthanised upon birth due to reaching HEP. In total three piglets were born still.

In case of sows vaccinated with ASFV-G-Δ9GL/ΔUK, all the live born piglets survived until the end of the study. One sow had 3 still born piglets while the other had none. The delivery was smooth and under normal circumstances. For the control animals, the delivery was under normal circumstances and all the live born animals survived until end of the study. The reproductive performance of sows in this study is presented in Table 2 herebeneath.

TABLE 2
reproductive performance of mother animals

| | Lv17/WB/ | ASFV-G-Δ9GL/ | |
|---|---|---|---|
| | Rie1-ΔCD | ΔUK | control |
| Total number of piglets | 26 | 41 | 43 |
| Normal piglets at birth | 0 | 37 | 39 |
| Weak piglets at birth | 23 | 1 | 0 |
| Dead piglets at birth | 3 | 3 | 4 |
| Alive piglets at end of trial | 0 | 34 | 28 |
| Loss of viable piglets | 100% | 10% | 35%* |

*8 of the 11 animals that did not reach the end of the study died of an accident (they got trapped under the sow). A corrected loss is thus 16%.

### Health Status of the Piglets

As none of the piglets born from sows vaccinated with Lv17/WB/Rie1-ΔCD were alive more than 1 day post farrow, the health status was only monitored for the other animals. The rectal temperatures of all piglets were normal throughout the experiment, and not different between the control and vaccinated animals. With regard to weight gain, piglets of the control sows showed on average a 3.7-fold increase from weight at birth, while in case of the vaccinated sows the piglets showed on average a 2.7-fold increase in weight.

The control piglets were all healthy throughout the experiment. Of the vaccinated animals, thirteen piglets born alive showed mild ASF-related clinical signs and one animal had moderate signs. ASFV infection was confirmed by PCR. A specific PCR performed on DNA extracted from blood of pregnant sows and their representative piglets indicate that the candidate vaccine strain was transmitted vertically from the pregnant sows to their piglets. Five piglets were viremic, 5 days after birth. Viremia reached its peak at 12 days after which it gradually declined. Notably, the viremic piglets remained healthy and alive until the end of the study.

### Conclusion

By comparison of the health status of the sows (and piglets born alive from these sows) with control sows, in particular since the loss of viable offspring is over 50%, it can be concluded that the Lv17/WB/Rie1-ΔCD strain is not safe for administration to pregnant swine, whereas ASFV-G-Δ9GL/ΔUK, not even leading to a loss in viable piglets higher than control (10% vs 35%/16%), is deemed safe for vaccinating pregnant swine.

### Example 3

The objective of this study was to provide confirmation that the ASF vaccine as used in Example 2, i.e. the MLV ASFV-G-Δ9GL/ΔUK, was indeed protective for the sows that received the vaccine during the third trimester of gestation. For this, three groups of animals were used: three sows in group 1, three sows in group 2 and 2 negative control animals.

The sows of group 1, which were vaccinated during the third trimester of gestation, were viraemic and indeed completely protected against a virulent challenge (data not provided), in line with what is known in the art regarding the protective nature of ASFV-G-Δ9GL/ΔUK. The sows of group 2 were not challenged and served as a negative control group, they survived until the end of the study.

1. A method for protecting a pregnant swine against an infection with African swine fever virus (ASFV), comprising administering to a pregnant swine a vaccine comprising live attenuated African swine fever virus Georgia 2007 (ASFV-G)-Δ9GL/ΔUK strain.

2. The method of claim 1, characterised in that the vaccine is administered to the pregnant swine in the second half of gestation.

3. The method of claim 1, wherein the vaccine is administered to the pregnant swine in the last third of gestation.

4. The method of claim 1, wherein the vaccine is administered to the pregnant swine in a period of 5-20 days before the expected date of farrowing of the pregnant swine.

5. The method of claim 1, wherein the vaccine is administered once during gestation or in a prime and boost regimen to the pregnant swine.

6. The method of claim 1, wherein the vaccine is administered intramuscularly, intradermally or orally.

7. The method of claim 1, wherein the vaccine is administered with a dose of the ASFV-G-Δ9GL/ΔUK strain of at least 10² TCID₅₀ per administration.

8. The method of claim 1, wherein the vaccine is administered with a dose of the ASFV-G-Δ9GL/ΔUK strain between 10² TCID₅₀ and 10⁶ TCID₅₀ per administration.

9. A live attenuated African swine fever virus Georgia 2007 (ASFV-G) Δ9GL/ΔUK strain for use in the manufacture of a vaccine for the protection of a pregnant swine against an infection with African swine fever virus.

10. (canceled)
